(19) United States
(12) Patent Application Publication
(10) Pub. No.: US 2026/0192636 A1
(43) Pub. Date: Jul. 9, 2026

(54) CAR AROMA DIFFUSER WITH SELECTABLE SCENT

(71) Applicant: DONGGUAN YIH TEH ELECTRIC PRODUCTS CO., LTD., DongGuan City (CN)

(72) Inventor: Ming Jen Hsiao, Miaoli County (TW)

(21) Appl. No.: 19/011,974

(22) Filed: Jan. 7, 2025

(51) Int. Cl. B60H 3/00

(52) U.S. Cl. CPC B60H 3/0035; B60H 2003/0042; B60H 2003/0064

(57) ABSTRACT

The car aroma diffuser with selectable scent of the present invention, when the function key is pressed, the first fan draws air through the first valve device into the first trough to guide the fragrance diffused by the first fragrance box to be output to the second valve device to the interior space of the car. By pressing the function key again, the first fragrance box will stop outputting scent and the second fan will start to draw air through the third valve device into the second trough to bring another scent from the second fragrance box to the fourth valve device to the interior of the car. Users can choose different scents, and the fragrance boxes are easy to replace to provide more choices. The fragrance boxes are filled with solid fragrance and will not leak and pollute the car.

### BACKGROUND OF THE INVENTION

### 1. Field of the Invention

The present invention relates to an aroma diffuser, and more particularly to a car aroma diffuser with selectable scent

### 2. Description of the Related Art

Conventional car aroma diffusers only provide a single fragrance and cannot provide multiple fragrance options, or it is inconvenient to replace or refill the fragrance. Such examples include U.S. 20210070148A1, U.S. Pat. No. 8,983,277B2, and U.S. Pat. No. 9,031,392B2 in the United States, which also use essential oils or liquid fragrances, which may leak and cause damage to the environment inside the car or raise safety concerns.

### SUMMARY OF THE INVENTION

In view of the above problems of the prior art, the present invention provides a car aroma diffuser with selectable scent, which can select different scents and fragrances or quickly and easily replace the fragrance box to provide more scent and fragrance choices.

The present invention also provides a car aroma diffuser with selectable scent, which is safe and will not leak fragrance in the car.

In some embodiments of the present invention, the car aroma diffuser with selectable scent comprises a bottom cover, a holder seat, a first fan, a first fragrance box, a second fan, a second fragrance box, a control circuit board, and a top cover. The holder seat comprises a first accommodating chamber located on one side thereof, a first bottom hole located on a bottom side of the first accommodating chamber, a first trough located on a front side thereof, a first valve device located on one side of the first trough, a second valve device located on an opposite side of the first trough, a first air inlet provided on an opposite side thereof and connected to the first bottom hole, a second accommodating chamber disposed in parallel to the first accommodating chamber, a second bottom hole provided at a bottom side of said second accommodating chamber, a third valve device provided at a front side of the second accommodating chamber, a second trough connected with one side thereof to the third valve device, a fourth valve device provided at an opposite side of the second trough, and a second air inlet disposed parallel to the first air inlet and connected to the second bottom hole. The first fan mounted in said first accommodating chamber. The first fragrance box detachably mounted in said first trough. The second fan mounted in said second accommodating chamber. The second fragrance box detachably mounted in said second trough. The control circuit board coupled to the holder seat, comprising a power supply unit and at least function key electrically connected to the first fan, the second fan, the first valve device, the second valve device, the third valve device and the fourth valve device. The top cover covers an upper side of the holder seat. The bottom cover covers an opposing lower side of said holder seat.

In some embodiments of the present invention, the fragrance boxes can emit various scents, including fragrances, deodorants, etc. The fragrance boxes store various fragrances, such as solid fragrances such as fragrance stones that use carriers to absorb fragrances. The fragrances will not leak out of the fragrance boxes and can be used safely in the car space without damaging the decoration and environment in the car.

By this, the control circuit board is electrically connected to a car power source, the indicator light of the power unit is on, the two fans are turned off in the power-on standby state, and the four valve devices are all turned off. The user can press the function key to select to turn on, for example, the first valve device and the second valve device. The first fan draws the airflow outside the first air inlet to pass through the first bottom hole and the first valve device. When the airflow enters the first trough, the odor emitted by the first fragrance box, such as fragrance or deodorant, is guided by the airflow and output from the second valve device to the interior space of the car to provide an environmental fragrance or eliminate odors in the car, bringing passengers and the driver a pleasant riding mood. The advantage of the present invention is that the user can change the scent at any time. The user can press the function key or another function key again to close the first valve device and the second valve device, and pause the first fan and the first fragrance box from outputting the scent while opening the third valve device and the fourth valve device, so that the second fan draws the airflow outside the second air inlet to pass through the second bottom hole and the third valve device. When the airflow enters the second trough, another fragrance, such as another fragrance or deodorant, emitted by the second fragrance box is guided by the airflow and output from the fourth valve device to the interior space of the car to provide an environmental fragrance in the car or eliminate odors or bad smells in the car. The use can choose to press the function key or another function key again to cycle through the scents of different fragrance boxes. The scent output mechanisms of the two fragrance boxes can be extended to multiple ones to expand more scent selection functions.

The advantages of the car aroma diffuser with selectable scent of the present invention are that the user can operate the function key to replace the fragrance boxes at any time to emit fragrance or smell. The fragrance boxes are easy to replace and provide users with more choices. The solid fragrance will not leak and pollute the car, providing a safe aroma diffuser for car use.

### BRIEF DESCRIPTION OF THE DRAWINGS

FIG. 1 is a three-dimensional external view of the car aroma diffuser with selectable scent of the present invention.

FIG. 2 is an exploded view of FIG. 1.

FIG. 3 is an exploded view of FIG. 1 from another viewing angle.

FIG. 4 is a top view of the car aroma diffuser with selectable scent of the present invention of FIG. 1.

FIG. 5 is a cross-sectional view taken along line A-A of FIG. 4.

FIG. 6 is a cross-sectional view taken along line B-B of FIG. 4.

FIG. 7 is an exploded view of FIG. 1 from another viewing angle.

FIG. 8 is a rear view of FIG. 1.

FIG. 9 is a cross-sectional view taken along line C-C of FIG. 4.

### DETAILED DESCRIPTION OF THE PREFERRED EMBODIMENT

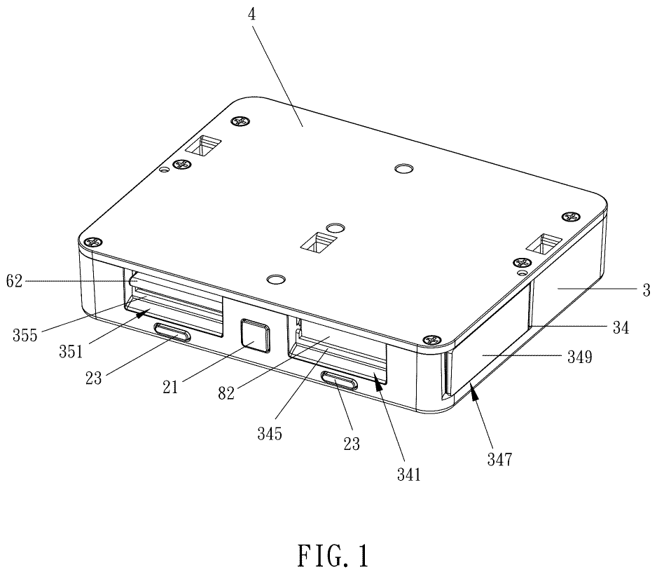

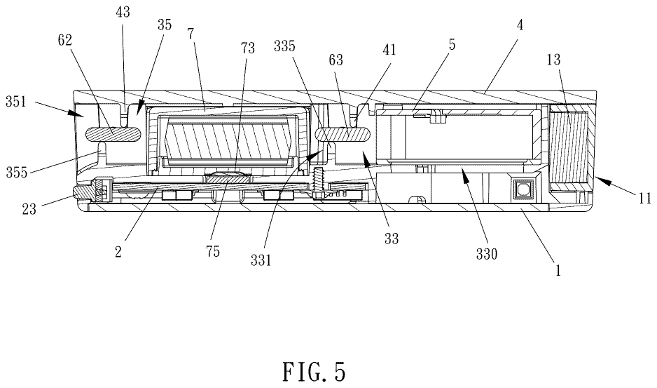

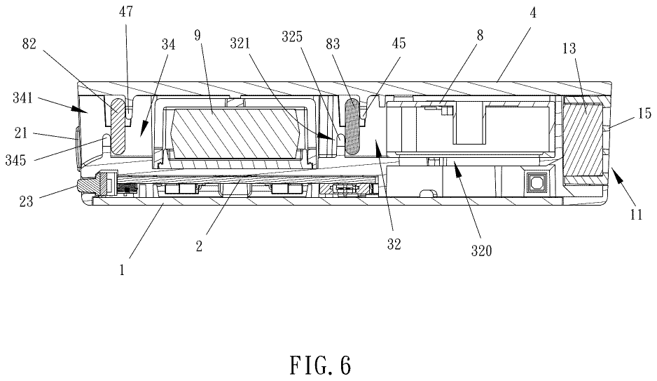

Please refer to FIGS. 1 to 6, some embodiments of the car aroma diffuser with selectable scent of the present invention are shown. The aroma diffuser comprises a bottom cover 1, a holder seat 3, a first fan 5, a first fragrance box 7, a second fan 8, a second fragrance box 9, a control circuit board 2, and a top cover 4. The holder seat 3 comprises a first accommodating chamber 33 on one side, a first bottom hole 330 on the bottom side, a first trough 35 on the front side, a first valve device 331 on one side of the first trough 35, a second valve device 351 on an opposite side of the first trough 35, and a first air inlet 31 provided on another side of the holder seat 3 and connected to the first bottom hole 330. The first fan 5 is coupled to the first accommodating chamber 33. The first fragrance box 7 is detachably coupled to the first trough 35. The holder seat 3 further comprises a second accommodating chamber 32 in a direction parallel to the first accommodating chamber 33, a second bottom hole 320 provided at the bottom side of the second accommodating chamber 32, a third valve device 321 provided at the front side of the second accommodating chamber 32, a second trough 34 connected with its one side to the third valve device 321, a fourth valve device 341 provided at another side of the second trough 34, and a second air inlet 30 disposed parallel to the first air inlet 31 and connected to the second bottom hole 320. The second fan 8 is coupled to the second accommodating chamber 32. The second fragrance box 9 is detachably coupled to the second trough 34. The control circuit board 2 is coupled to another side of the holder seat 3. The control circuit board 2 comprises a power supply unit 21 and at least one, for example, two function keys 23, which are electrically connected to the first fan 5, the second fan 8, the first valve device, the second valve device, the third valve device and the fourth valve device. The top cover 4 covers the top side of the holder seat 3, and the bottom cover 1 covers the bottom side of the holder seat 3.

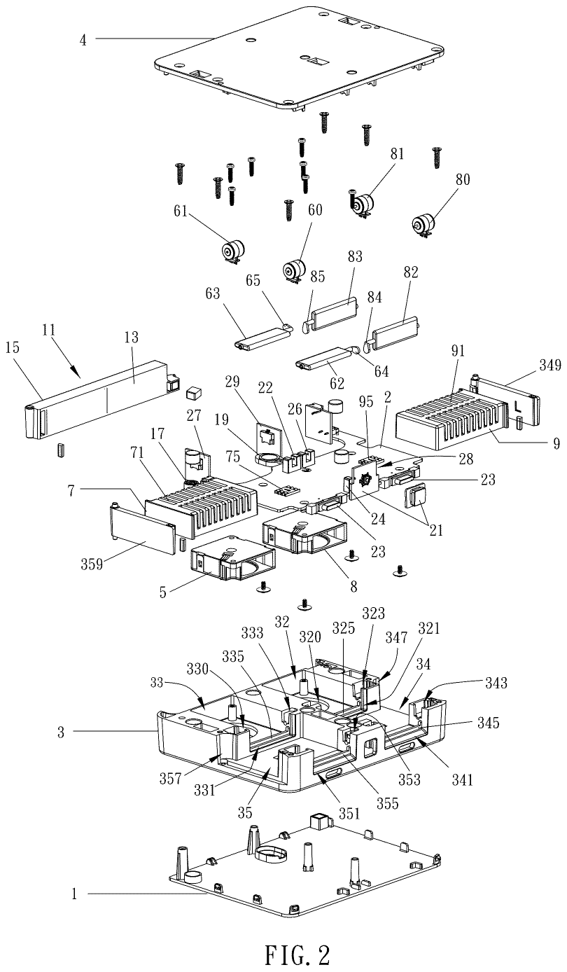

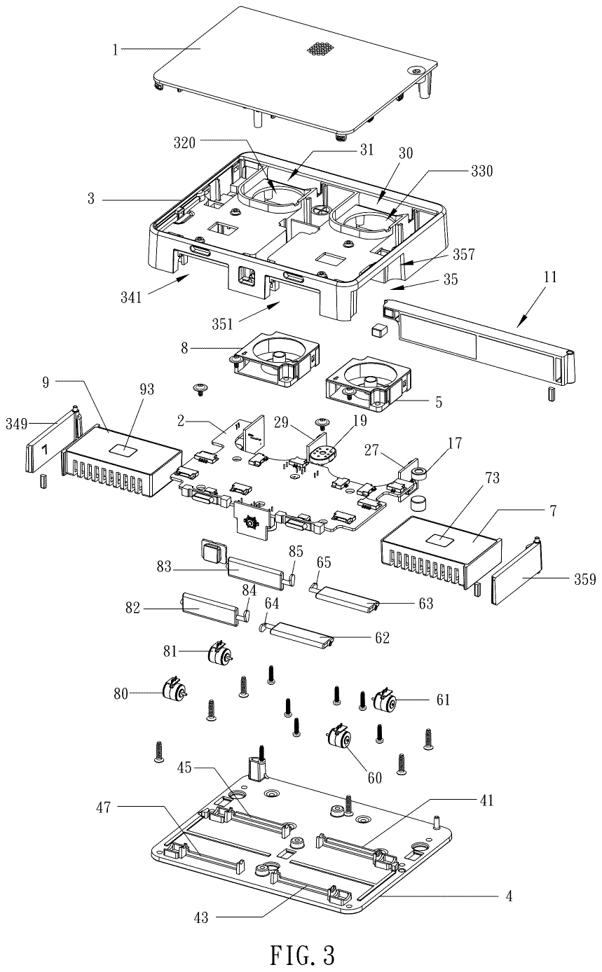

Referring to FIGS. 2 and 3, in some embodiments of the present invention of the car aroma diffuser with selectable scent, the first fragrance box 7 or the second fragrance box 9 is used to emit various scents such as fragrance. The first fragrance box 7 comprises a first fragrance diffusion hole 71. The second fragrance box 9 comprises a second fragrance diffusion hole 91. The first fragrance box 7 or the second fragrance box 9 stores a solid fragrance of perfume or deodorant absorbed by a carrier, and can emit fragrance or odor from the first fragrance diffusion hole 71 or the second fragrance diffusion hole 91. When the airflow of the first fan 5 enters the first trough 35, the airflow can enter the first fragrance box 7 from one side of the first fragrance diffusion hole 71, and then flow out from another side, so that the smell of the first fragrance box 7 is guided by the airflow and output to the second valve device 351 into the car interior space. When the airflow of the second fan 8 enters the second trough 34, the airflow can enter the second fragrance box 9 from one side of the second fragrance diffusion hole 91, and then flow out from another side, so that another fragrance emitted by the second fragrance box 9 is guided by the airflow and output from the fourth valve device 341 to the interior space of the car. In some embodiments, solid fragrances, such as fragrance stones, pumice stones storing fragrances, or various solidified fragrances of clay, plastic, wood, stone, polymer foam, paper sticks, fiber sticks or fabrics absorbing fragrances or essential oils, can be emitted from the first fragrance box 7 or the second fragrance box 9 by evaporation, volatilization, and the fragrance will not leak out of the fragrance box and can be used safely in the car without damaging the interior decoration and environment.

Thus, the control circuit board 2 is electrically connected to a car power source (e.g. 12V), the indicator light of the power unit 21 is on, the two fans are turned off in the power-on standby state, and the four valve devices are all turned off. The user can press the function key 23 to select to turn on, for example, the first valve device 331 and the second valve device 351. The first fan 5 draws the airflow outside the first air inlet 31 to pass through the first bottom hole 330 and the first valve device 331. When the airflow enters the first trough 35, the odor emitted by the first fragrance box 7, such as fragrance or deodorant, is guided by the airflow and output from the second valve device 351 to the interior space of the car to provide an environmental fragrance or eliminate odors in the car, bringing passengers and the driver a pleasant riding mood.

The advantage of the present invention is that the user can change the scent at any time. The user can press the function key 23 or another function key 23 again to close the first valve device 331 and the second valve device 351, and pause the first fan 5 and the first fragrance box 7 from outputting the scent while opening the third valve device 321 and the fourth valve device 341, so that the second fan 8 draws the airflow outside the second air inlet 30 to pass through the second bottom hole 320 and the third valve device 321. When the airflow enters the second trough 34, another fragrance, such as another fragrance or deodorant, emitted by the second fragrance box 9 is guided by the airflow and output from the fourth valve device 341 to the interior space of the car to provide an environmental fragrance in the car or eliminate odors or bad smells in the car. You can choose to press the function key 23 or another function key 23 again to cycle through the scents of different fragrance boxes. The scent output mechanisms of the two fragrance boxes can be extended to multiple ones to expand more scent selection functions.

Users can operate the function keys to change the fragrance box at any time to emit fragrance or smell, providing users with more flavor choices. Solid fragrance will not leak and pollute the car, providing a safe aroma diffuser for car use.

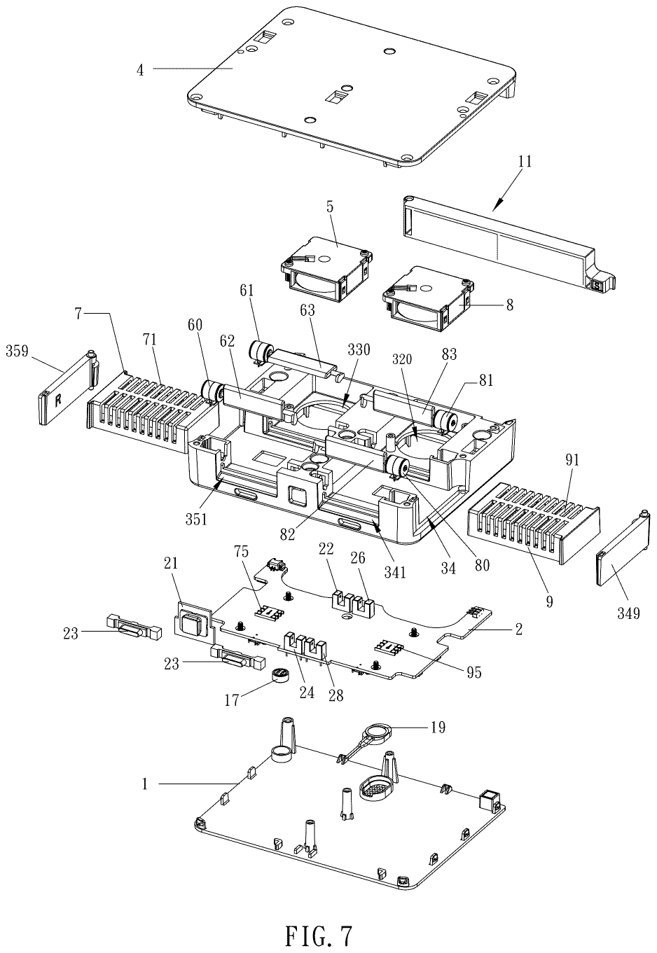

Referring to FIGS. 2 to 7, in some embodiments of the present invention of the car aroma diffuser with selectable scent, the first valve device 331 comprises a first hole 333 on one side, a first driving device 61, and a first valve flap 63. A first baffle 65 protrudes from one side of the first valve flap 63, and another side of the first valve flap 63 is connected to the first driving device 61. The first driving device 61 and the first valve flap 63 are combined with the first valve device 331, and the first baffle 65 is located in the first valve hole 333.

The second valve device 351 comprises a second valve hole 353 on one side, a second driving device 60, and a second valve flap 62. A second baffle 64 protrudes from one side of the second valve flap 62, and another side of the second baffle 64 is connected to the second driving device 60. The second driving device 60 and the second valve flap 62 are combined with the second valve device 351, and the second baffle 64 is located in the second valve hole 353.

The third valve device 321 comprises a third valve hole 323 on one side, a third driving device 81, and a third valve flap 83. A third baffle 85 protrudes from one side of a third valve flap 83, and another side of the third baffle 85 is connected to the third driving device 81. The third driving device 81 and the third valve flap 83 are combined with the third valve device 321, and the third baffle 85 is located in the third valve hole 323.

The fourth valve device 341 comprises a fourth valve hole 343 on one side, a fourth driving device 80, and a fourth valve flap 82. A fourth baffle 84 protrudes from one side of the fourth valve flap 82, and another side of the fourth baffle 84 is connected to the fourth driving device 80. The fourth driving device 80 and the four valve flap 82 are combined with the fourth valve device 341, and the fourth baffle 84 is located in the fourth valve hole 343.

The control circuit board 2 further comprises a first thru-beam switch 22 coupled to the first valve hole 333. The first valve flap 63 is driven by the first driving device 61 to rotate a certain angle to open and close the first valve device 331 and link the first baffle 65 to rotate to block or leave and connect the first thru-beam switch 22.

A second thru-beam switch 24 is coupled to the second valve hole 353. The second valve flap 62 is driven by the second driving device 60 to rotate a certain angle to open and close the second valve device 351 and link the second baffle 64 to rotate to block or leave and connect the second thru-beam switch 24.

A third thru-beam switch 26 is coupled to the third valve hole 323. The third valve flap 83 is driven by the third driving device 81 to rotate a certain angle to open and close the third valve device 321 and link the third baffle 85 to rotate to block or leave and connect the third thru-beam switch 26.

A fourth thru-beam switch 28 is coupled to the fourth valve hole 343. The fourth valve flap 82 is driven by the fourth driving device 80 to rotate a certain angle to open and close the fourth valve device 341 and link the fourth baffle 84 to rotate to block or leave and connect the fourth thru-beam switch 28.

Referring to FIGS. 1-7, in some embodiments of the present invention of the car aroma diffuser with selectable scent, the first thru-beam switch 22, the second thru-beam switch 24, the third thru-beam switch 26 and the fourth thru-beam switch 28 are U-shaped LED light source switches. The first baffle 65 is used to rotate to block or leave and connect the LED light source in the first thru-beam switch 22 U-shaped structure. The second baffle 64 is used to rotate to block or leave and connect the LED light source in the second thru-beam switch 24 U-shaped structure. The third baffle 85 is used to rotate to block or leave and connect the LED light source in the third thru-beam switch 26 U-shaped structure. The fourth baffle 84 is used to rotate to block or leave and connect the LED light source in the fourth thru-beam switch 28 U-shaped structure.

In some embodiments of the car aroma diffuser with selectable scent of the present invention, the first driving device 61, the second driving device 60, the third driving device 80 and the fourth driving device 81 can be, for example, a stepping motor or a motor combined with a transmission device, such as a connecting rod combined with a gear set, which can be understood and implemented by ordinary technicians in the field.

In some embodiments of the car aroma diffuser with selectable scent of the present invention, the user can press the function key 23 to select to turn on the first driving device 61 (such as a stepper motor) to drive the first valve flap 63, which rotates to 0 degrees horizontally to open the first valve device 331, and at the same time links the first baffle 65 to leave the U-shaped groove of the first thru-beam switch 22 to connect the light sources on both sides. The second driving device 60 (such as a stepper motor) drives the second valve flap 62 synchronously, to rotate to a horizontal position of 0 degrees to open the second valve device 351, and simultaneously link the second baffle 64 to leave the U-shaped groove of the second thru-beam switch 24 and connect the light sources on both sides, so as to turn on the first fan 5 and the control circuit board 2, so that the control circuit board 2 starts recording various information of the aroma diffuser and controls the lighting of the first fragrance box 7 in use. The first fan 5 operates the airflow to pass through the first valve device 331 to the first trough 35, driving the first fragrance box 7 to emit the fragrance through the second valve device 351 and output it to the vehicle.

If the user wants to select another scent or fragrance, he can press the function key 23 or another function key 23 again to activate the first valve flap 63 to rotate 90 degrees vertically to close the first valve device 331, and at the same time, the first baffle 65 is linked to block the light source in the U-shaped groove of the first thru-beam switch 22, and the second valve flap 62 rotates 90 degrees to vertically close the second valve device 351, and at the same time, the second baffle 64 is linked to block the light source in the U-shaped groove of the first thru-beam switch 22. While pausing the first fan 5 and the first fragrance box 7 to output the scent, the third driving device 81 (such as a stepping motor) is turned on to drive the third valve flap 83 to rotate to a horizontal position of 0 degrees to open the third valve device 321, and at the same time, the third baffle 85 is linked to leave the U-shaped groove of the third thru-beam switch 26 to connect the light sources on both sides. The fourth driving device 80 (such as a stepper motor) drives the fourth valve flap 82 to rotate horizontally 0 degrees to open the fourth valve device 341, and the fourth baffle 84 is linked to leave the U-shaped groove of the fourth thru-beam switch 28 to connect the two side light sources, so as to turn on the second fan 8 and the control circuit board 2, so that the control circuit board 2 starts recording various information of the aroma diffuser and controls the lighting of the second fragrance box 9 in use. The second fan 8 drives the airflow to pass through the third valve device 321 to the second trough 34, driving the second fragrance box 9 to emit another scent or fragrance through the fourth valve device 341 to the interior space of the car, thereby quickly achieving the scent change selection. The user can press another function key 23 to cycle through the above implementation procedures to select different fragrance boxes or scents in the car.

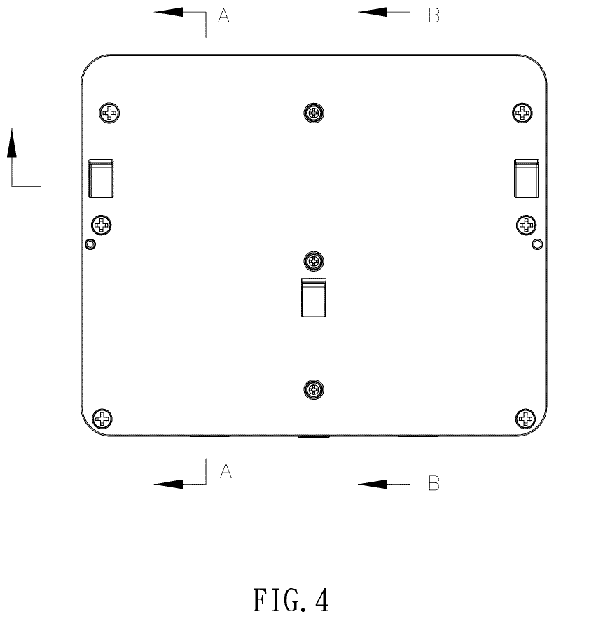

Referring to FIGS. 4 to 7, in some embodiments of the present invention of the car aroma diffuser with selectable scent, the first valve device 331 further comprises a first threshold 335 protruding from the bottom side, and the top cover 4 further comprises a first limit plate 41 protruding from the inner surface thereof. The first valve flap 63 is driven by the first driving device 61 to rotate horizontally to 0 degrees between the first threshold 335 and the first limit plate 41 to open the first valve device 331, and reciprocated to vertically rotate to 90 degrees between the first threshold 335 and the first limit plate 41 to close the first valve device 331.

The second valve device 351 further comprises a second threshold 355 protruding from the bottom, and the top cover 4 further comprises a second limit plate 43 protruding from the inner surface thereof. The second valve flap 62 is driven by the second driving device 60 to rotate horizontally to 0 degrees between the second threshold 355 and the second limit plate 43 to open the second valve device 351, and reciprocated to vertically rotate to 90 degrees between the second threshold 355 and the second limit plate 43 to close the second valve device 351.

A third threshold 325 protrudes from the bottom of the third valve device 321, and a third limit plate 45 protrudes from the inner surface of the top cover 4. The third valve flap 83 is driven by the third driving device 81 to rotate horizontally to 0 degrees between the third threshold 325 and the third limit plate 45 to open the third valve device 321, and reciprocated to vertically rotate to 90 degrees between the third threshold 325 and the third limit plate 45 to close the third valve device 321.

The fourth valve device 341 further comprises a fourth threshold 345 protruding from the bottom side, and the top cover 4 further comprises a fourth limit plate 47 protruding from the inner surface thereof. The fourth valve flap 82 is driven by the fourth driving device 80 to rotate horizontally to 0 degrees between the fourth threshold 345 and the fourth limit plate 47 to open the fourth valve device 341, and reciprocated to vertically rotate to 90 degrees between the fourth threshold 345 and the fourth limit plate 47 to close the fourth valve device 341.

Referring to FIGS. 2-6, in some embodiments of the present invention of the car aroma diffuser with selectable scent, the control circuit board 2 further comprises a first UVC 27 and a second UVC 29 for sterilizing the airflow inhaled by the fan with UV light. The first UVC 27 is adjacent to the first bottom hole 330 or the first air inlet 31, and the second UVC 29 is adjacent to the second bottom hole 320 or the second air inlet 30. When the function key 23 is selected to open the first valve device 331 and the second valve device 351, the first UVC 27 is turned on at the same time, and the airflow input by the first fan 5 is sterilized with UV light to provide clean airflow mixed with the smell emitted by the first fragrance box 7, such as fragrance, to output clean fragrance, thereby improving the air quality in the car. When the function key 23 is selected to open the third valve device 321 and the fourth valve device 341, the second UVC 29 is turned on at the same time to sterilize the airflow input by the second fan 8 with UV light to provide clean airflow mixed with the smell emitted by the second fragrance box 9, such as fragrance, to output a clean aroma.

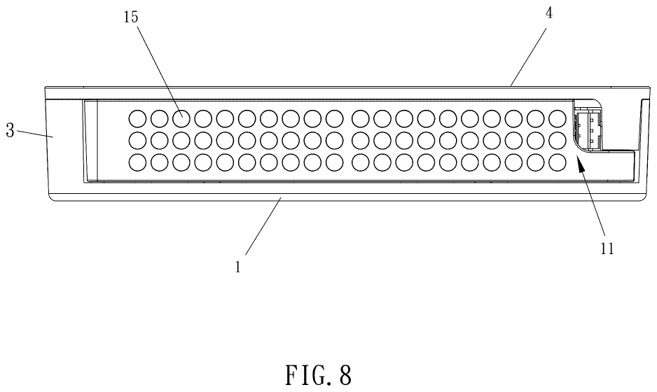

Referring to FIGS. 4-8, in some embodiments of the present invention of the car aroma diffuser with selectable scent, the car aroma diffuser further comprises a filter 11. The filter 11 is combined with the outer sides of the first air inlet 31 and the second air inlet 30. The filter 11 comprises a filter material 13 and a perforated plate 15. The filter material 13 is combined with the perforated plate 15 to form the filter 11. The airflow attracted by the first fan 5 or the second fan 8 first passes through the perforated plate 15 of the filter 11 and the filter material 13 such as filter cotton to filter the polluted air. The filter material includes, for example, any one of filter cotton, filter mesh, cloth, fiber cloth, non-woven fabric or activated carbon.

Referring to FIGS. 1-3 and 7, in some embodiments of the present invention of the car aroma diffuser with selectable scent, the first trough 35 further comprises a first door 357 and a first door panel 359. One side of the first door panel 359 can be movably combined with one side of the first door 357 so that the first door panel 359 can conveniently open or close the first door 359 to quickly and conveniently replace the first fragrance box 7. The second trough 34 further comprises a second door 347 and a second door panel 349. One side of the second door panel 349 can be movably connected to the first door 357 so that the second door panel 349 can conveniently open or close the second door 347 to quickly and conveniently replace the second fragrance box 9. In some embodiments, the door and the door panel are equipped with magnets or magnetic materials, whereby the other side of the first door panel 359 is used to quickly open or close the first door 357 by magnetic attraction, and the other side of the second door panel 349 is used to quickly open or close the second door 347 by magnetic attraction.

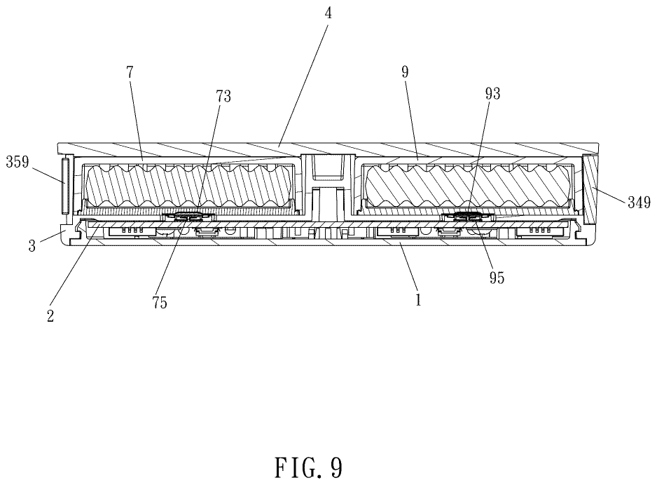

Referring to FIGS. 2, 3 and 9, in some embodiments of the present invention of the car aroma diffuser with selectable scent, the first fragrance box 7 further comprises a first identification chip 73, and the second fragrance box 9 further comprises a second identification chip 93. The control circuit board 2 further comprises a first sensor 75 and a second sensor 95. The first sensor 75 is used to read the information of the first identification chip 73, and the second sensor 95 is used to read the information of the second identification chip 93. The first identification chip 73 and the second identification chip 93 contain any information such as odor, concentration, fragrance, deodorant usage record, or fragrance box source. When the first sensor 75 reads the first identification chip 73, or the second sensor 95 reads the second identification chip 93, and it is not the identification chip of the aroma diffuser, the aroma diffuser will not turn on. The aroma diffuser can only turn on when the matching identification chip is read.

Referring to FIGS. 2, 3 and 7, in some embodiments of the present invention of the car aroma diffuser with selectable scent, the control circuit board 2 further comprises an APP control function and is connected to a microphone 17 and a speaker 19 for voice switching and voice control input functions, or selecting a fragrance box or fragrance concentration, etc.

What the invention claimed is:

1. A car aroma diffuser with selectable scent, comprising:
a holder seat comprising a first accommodating chamber located on one side thereof, a first bottom hole located on a bottom side of said first accommodating chamber, a first trough located on a front side thereof, a first valve device located on one side of said first trough, a second valve device located on an opposite side of said first trough, a first air inlet provided on an opposite side thereof and connected to said first bottom hole, a second accommodating chamber disposed in parallel to said first accommodating chamber, a second bottom hole provided at a bottom side of said second accommodating chamber, a third valve device provided at a front side of said second accommodating chamber, a second trough connected with one side thereof to said third valve device, a fourth valve device provided at an opposite side of said second trough, and a second air inlet disposed parallel to said first air inlet and connected to said second bottom hole;
a first fan mounted in said first accommodating chamber;
a first fragrance box detachably mounted in said first trough;
a second fan mounted in said second accommodating chamber;
a second fragrance box detachably mounted in said second trough;
a control circuit board coupled to said holder seat, said control circuit board comprising a power supply unit and at least function key electrically connected to said first fan, said second fan, said first valve device, said second valve device, said third valve device and said fourth valve device;
a top cover covering an upper side of said holder seat; and
a bottom cover covering an opposing lower side of said holder seat.

2. The car aroma diffuser with selectable scent as claimed in claim 1, wherein said first fragrance box and second fragrance box are used to emit different scents; said first fragrance box comprises a first fragrance diffusion hole, and said second fragrance box comprises a second fragrance diffusion hole; said first fragrance box and said second fragrance box respectively store solid fragrances using carriers to absorb fragrances or deodorants, including fragrance stones, pumice stones storing fragrances, or various solidified fragrances of clay, plastic, wood, stone, polymer foam, paper sticks, fiber sticks or fabrics absorbing fragrances or essential oils.

3. The car aroma diffuser with selectable scent as claimed in claim 1, wherein said control circuit board is used to electrically connect to a car power source, so that an user is capable of pressing one of said at least one function key to turn on said first fan and said first valve device and said second valve device; when said first fan is turned on, said first fan draws the airflow outside said first air inlet to pass through said first bottom hole and said first valve device into said first trough; when the airflow enters said first trough, the fragrance emitted by said first fragrance box is guided by the airflow and output to said second valve device into the interior space of the car carrying the car aroma diffuser, then the user is capable of pressing another one of said at least one function key to turn off said first fan and said first valve device and said second valve device, and pause said first fragrance box to output fragrance while turning on said second fan, said third valve device and said fourth valve device, so that said second fan draws the airflow outside said second air inlet to pass through said second bottom hole and said third valve device into said second trough, and when the airflow enters said second trough, another fragrance emitted by said second fragrance box is guided by the airflow and output from said fourth valve device to the interior space of the car.

4. The car aroma diffuser with selectable scent as claimed in claim 1, wherein:
said first valve device comprises a first valve hole located on one side thereof, a first driving device and a first valve flap, said first valve flap having one side thereof protruded with a first baffle and an opposite side thereof connected to said first driving device, said first driving device and said first valve flap being combined with said first valve device, and said first baffle being located in said first valve hole;
said second valve device comprises a second valve hole located on one side thereof, a second driving device and a second valve flap, said second valve flap having one side thereof protruded with a second baffle and an opposite side thereof connected to said second driving device, said second driving device and said second valve flap being combined with said second valve device, and said second baffle being located in said second valve hole;
said third valve device comprises a third valve hole located on one side thereof, a third driving device and a third valve flap, said third valve flap having one side thereof protruded with a third baffle and an opposite side thereof connected to said third driving device, said third driving device and said third valve flap being combined with said third valve device, and said third baffle being located in said third valve hole;
said fourth valve device comprises a fourth valve hole located on one side thereof, a fourth driving device and a fourth valve flap, said fourth valve flap having one side thereof protruded with a fourth baffle and an opposite side thereof connected to said fourth driving device, said fourth driving device and said fourth valve flap being combined with said fourth valve device, and said fourth baffle being located in said fourth valve hole;
said control circuit board further comprises a first thru-beam switch coupled to said first valve hole, a second thru-beam switch coupled to said second valve hole, a third thru-beam switch coupled to said third valve hole, and a fourth thru-beam switch coupled to said fourth valve hole,
said first valve flap is driven by said first driving device to rotate by a certain angle to open and close said first valve device and link said first baffle to rotate to block or leave and connect to said first thru-beam switch;
said second valve flap is driven by said second driving device to rotate at a certain angle to open and close said second valve device and to link said second baffle to rotate to block or leave and connect to said second thru-beam switch;
said third valve flap is driven by said third driving device to rotate by a certain angle to open and close said third valve device and link said third baffle to rotate to block or leave and connect to said third thru-beam switch;
said fourth valve flap is driven by said fourth driving device to rotate by a certain angle to open and close said fourth valve device and link said fourth baffle to rotate to block or leave and connect to said fourth thru-beam switch.

5. The car aroma diffuser with selectable scent as claimed in claim 4, wherein said first thru-beam switch, said second thru-beam switch, said third thru-beam switch and said fourth thru-beam switch are U-shaped LED light source switches.

6. The car aroma diffuser with selectable scent as claimed in claim 5, wherein when the user press said at least one function key to select to turn on said first driving device to drive said first valve flap, which rotates to 0 degrees horizontally to open said first valve device, and at the same time links said first baffle to leave an U-shaped groove of said first thru-beam switch to connect light sources on both sides, and said second driving device drives said second valve flap synchronously, to rotate to a horizontal position of 0 degrees to open said second valve device, and simultaneously link said second baffle to leave an U-shaped groove of said second thru-beam switch and connect light sources on both sides, so as to turn on said first fan and said control circuit board, so that said control circuit board starts recording various information of the aroma diffuser and controls the lighting of said first fragrance box in use, and said first fan drives the airflow to pass through said first valve device to said first trough, driving said first fragrance box to emit the fragrance through said second valve device and output the fragrance into the interior space of car; when the user press said at least one function key again to activate said first valve flap to rotate 90 degrees vertically to close said first valve device, and at the same time, said first baffle is linked to block the light source in the U-shaped groove of said first thru-beam switch, and said second valve flap is rotated 90 degrees vertically to close said second valve device, at the same time, said second baffle is linked to block the light source in the U-shaped groove of said first thru-beam switch, and said first fan and said first fragrance box are temporarily stopped from outputting fragrance, at the same time, said third driving device is turned on to drive said third valve flap to rotate to 0 degrees horizontally to open said third valve device, and said third baffle is linked to leave the U-shaped groove of said third thru-beam switch to connect the light sources on both sides, and said fourth driving device is synchronized to drive said fourth valve flap to rotate horizontally to 0 degrees to open said fourth valve device, and said fourth baffle is linked to leave the U-shaped groove of said fourth thru-beam switch to connect the light sources on both sides and to turn on said second fan and said control circuit board, so that said control circuit board starts recording various aroma diffuser information and controlling the lighting of said second fragrance box in use, and by this, said second fan drives the airflow to pass through said third valve device to said second trough, driving said second fragrance box to emit another scent or fragrance through said fourth valve device into the interior space of the car, thereby quickly achieving the scent change option.

7. The car aroma diffuser with selectable scent as claimed in claim 6, wherein said first driving device, said second driving device, said third driving device and said fourth driving device are selectively composed of a stepping motor or a motor combined with a transmission device.

8. The car aroma diffuser with selectable scent as claimed in claim 4, wherein said first valve device further comprises a first threshold protruding from the bottom side, and the top cover further comprises a first limit plate protruding from the inner surface thereof; the first valve flap is driven by the first driving device to rotate horizontally to 0 degrees between the first threshold and the first limit plate to open the first valve device, and reciprocated to vertically rotate to 90 degrees between the first threshold and the first limit plate to close the first valve device;
the second valve device further comprises a second threshold protruding from the bottom, and the top cover further comprises a second limit plate protruding from the inner surface thereof; the second valve flap is driven by the second driving device to rotate horizontally to 0 degrees between the second threshold and the second limit plate to open the second valve device, and reciprocated to vertically rotate to 90 degrees between the second threshold and the second limit plate to close the second valve device;
a third threshold protrudes from the bottom of the third valve device, and a third limit plate protrudes from the inner surface of the top cover; the third valve flap is driven by the third driving device to rotate horizontally to 0 degrees between the third threshold and the third limit plate to open the third valve device, and reciprocated to vertically rotate to 90 degrees between the third threshold and the third limit plate to close the third valve device;
the fourth valve device further comprises a fourth threshold protruding from the bottom side, and the top cover further comprises a fourth limit plate protruding from the inner surface thereof; the fourth valve flap is driven by the fourth driving device to rotate horizontally to 0 degrees between the fourth threshold and the fourth limit plate to open the fourth valve device, and reciprocated to vertically rotate to 90 degrees between the fourth threshold and the fourth limit plate to close the fourth valve device.

9. The car aroma diffuser with selectable scent as claimed in claim 1, wherein said control circuit board further comprises a first UVC and a second UVC used to sterilize the airflow inhaled by said first fan and said second fan with UV light respectively, said first UVC being disposed adjacent to one of said first bottom hole and said first air inlet, said second UVC being disposed adjacent to one of said second bottom hole and said second air inlet.

10. The car aroma diffuser with selectable scent as claimed in claim 1, further comprising a filter combined with the outside of said first air inlet and said second air inlet.

11. The car aroma diffuser with selectable scent as claimed in claim 10, wherein said filter comprises a filter material and a perforated plate, said filter material being combined with said perforated plate to form said filter.

12. The car aroma diffuser with selectable scent as claimed in claim 1, wherein said first trough further comprises a first door and a first door panel, said first door panel having one side thereof movably connected to one side of said first door; said second trough further comprises a second door and a second door panel, said second door panel having one side thereof movably connected to one side of said second door.

13. The car aroma diffuser with selectable scent as claimed in claim 12, wherein said first door panel has an opposite side thereof used for magnetically opening or closing said first door quickly, and said second door panel has an opposite side thereof used for magnetically opening or closing said second door quickly.

14. The car aroma diffuser with selectable scent as claimed in claim 1, wherein said first fragrance box further comprises a first identification chip; second fragrance box further comprises a second identification chip; said control circuit board further comprises a first sensor and a second sensor, said first sensor being used to read the information of said first identification chip, said second sensor being used to read the information of said second identification chip.
